(19) United States
(12) Patent Application Publication
(10) Pub. No.: US 2015/0328268 A1
(43) Pub. Date: Nov. 19, 2015

(54) Marine Plants Extract for Wound Healing

(71) Applicant: BASF Corporation, Florham Park, NJ (US)

(72) Inventors: Liliana Craciun, Carmel, NY (US); Manasi M. Chavan, Stony Brook, NY (US)

(73) Assignee: BASF Corporation, Florham Park, NJ (US)

(21) Appl. No.: 14/700,326

(22) Filed: Apr. 30, 2015

Related U.S. Application Data
(60) Provisional application No. 61/993,008, filed on May 14, 2014.

(51) Int. Cl. A61K 36/03; A61K 45/06

(52) U.S. Cl. CPC A61K 36/03; A61K 45/06

(57) ABSTRACT

This application is directed to a method of promoting wound healing comprising administering to a patient in need of such treatment an effective amount of Fucus vesiculosus extract. The Fucus visiculosus extract promotes wound angiogenesis, the process by which new blood vessels grow into tissues forming capillaries. Thus the extract may be highly advantageous in promoting healing of chronic wounds such venous, arterial, neuropathic, or pressure wounds.

This application takes the benefit of U.S. Ser. No. 61/993008, filed on May 14, 2014, herein incorporated entirely by reference.

### FIELD OF INVENTION

This application is directed to a method of promoting wound healing comprising administering to a patient in need of such treatment an effective amount of Fucus vesiculosus extract. The manner of administering is via topical application directly to the wound site or via a bandage or membrane which releases the extract to the wound site. The Fucus visiculosus extract appears to promote wound angiogenesis, the process by which new blood vessels grow into tissues forming capillaries. Thus the extract may be highly advantageous in promoting healing of chronic wounds such venous, arterial, neuropathic, or pressure wounds.

### BACKGROUND

Normally, wounds are expected to heal within a 2 week period through a complex systemic cascade of events that includes inflammation, neovascularization, collagen synthesis, granulation tissue formation, re-epithelialization, and wound remodeling. This multi-step process requires the interaction of molecules such as growth factors, cytokines and proteases with many cell types and their extracellular matrix. When, despite optimum wound care, the healing process is halted at any of these stages, the wound does not heal and becomes chronic. A chronic wound may be defined as one that has not adequately re-epithelialized within 6-8 weeks. Common types of chronic wounds include venous, arterial, neuropathic, or pressure wounds.

Clinically, non-healing wounds include pressure sores, venous and arterial ulcers, and diabetic foot ulcers. The pathogenesis of these wounds is not fully understood. They encompass abnormalities in the healing processes of inflammation, cell migration and remodeling.

Current treatments for chronic wounds are costly and only moderately effective. They include medicated paste and bandages, advanced dressings, collagen-based products, tissue engineered skin substitutes, hyperbaric oxygen, negative pressure therapy, compression therapy, or natural products such as honey and tea tree oil, where wound healing is promoted by targeting individual phases of the wound repair.

However, healing of the wound will not proceed unless new, functioning blood vessels are present to supply oxygen and nutrients to the injured tissue. Under normal conditions, a tissue cannot grow beyond 1-2 mm in diameter without neovascularization, the distance being limited by the diffusion of oxygen and metabolites necessary for tissue regeneration. During angiogenesis capillary sprouts invade the fibrin/fibronectin-rich wound clot and within a few days organize into a microvascular network throughout the granulation tissue.

This vessel repair and wound angiogenesis, the process by which new blood vessels grow into tissues forming capillaries, is critical to all stages of wound healing and in particular to the proliferative phase. By supplying nutrients and oxygen to the active cells at the wound site, angiogenesis stimulates repair and supports the growth of new, healthy tissue. Healing of any skin wound other than the most superficial cannot occur without angiogenesis. Not only does any damaged vasculature need to be repaired, but the increased local cell activity necessary for healing requires an increased supply of nutrients from the bloodstream.

The present applicants have found that an extract of marine algae, Fucus vesiculosus, and to compositions comprising the extract, promote endothelial cell migration, angiogenesis and the in-growth of new blood vessels. The exceptional pro-angiogenic properties of the Fucus vesiculosus extract make it useful for applications in wound healing.

### SUMMARY OF THE INVENTION

The present application is directed to a method of promoting wound healing comprising administering to a subject in need thereof an effective amount of a composition comprising Fucus vesiculosus extract.

Furthermore, the application is directed to a wound care product comprising an effective amount of a composition comprising Fucus vesiculosus extract, wherein the wound care product is a bandage, a membrane, a dressing, synthetic or biological hydrogels, hydrocolloids, films, foams or skin substitutes.

### BRIEF DESCRIPTION OF THE DRAWINGS

FIG. 1. Time-lapse imaging of the migratory response of HMVEC cells to: (a) 1% FBS control; (b) FGF (10 ng/mL in media); (c) Fucus vesiculosus extract 5 mg/mL; (d) Fucus vesiculosus extract 20 mg/mL; and (e) Fucus vesiculosus extract 30 mg/mL in media.

FIG. 2. The rate of wound closure of mechanically injured HMVEC cells in the presence of Fucus vesiculosus extract at concentrations of 1-30 mg/mL. FGF (10 ng/mL) is employed as positive control, and 1% FBS is used as reference.

FIG. 3. The percent wound closure over time of mechanically injured HMVEC cells in the presence of Fucus vesiculosus extract at concentrations of 1-30 mg/mL. FGF (10 ng/mL) is employed as positive control, and 1% FBS is used as reference.

FIG. 4. Time-lapse imaging of the angiogenic response of BREC cells to: (a) 1% BCS; (b) FGF (10 ng/mL in media); (c) Fucus vesiculosus extract 30 mg/mL; and (d) Fucus vesiculosus extract 0.25mg/mL.

### DETAILED DESCRIPTION OF THE INVENTION

Fucus vesiculosusl

Fucus vesiculosus, also commonly known as bladderwrack or rock weed, is a species of temperate algae naturally found in littoral/lower intertidal zones along the coastlines of the Atlantic Oceans. It can also found in some Pacific ocean regions as well as the North Sea and Baltic Sea. The Fucus vesiculosus may be naturally occurring (i.e., “wild”) or cultivated.

Fucus vesiculosus is a marine organism rich in sources of structurally novel and biologically active metabolites with potential in health applications. Their bioactive components include polyphenols, peptides, and polysaccharides.

Extracts of Fucus vesiculosus are known for use as a cosmetic active ingredient as in co-pending U.S. application Ser. No. 14/077,934. It is believed the extract stimulates Heme oxygenase which in turn, stimulates the scavenging of heme thereby reducing the appearance of dark circles under eyes.

Most abundant in brown algae is a fucoidan polysaccharide, a complex sulfated polysaccharide mainly found in the cell-wall matrix. The main unit in fucoidan is L-fucose, a hexose deoxy sugar with the chemical formula C₆H₁₂O₅, with small quantities of D-galactose, D-mannose, D-xylose, and uronic acid, and several percentages of sulfate ester groups.

Fucoidans exhibit interesting biological properties, such as antioxidant, immunomodulatory, antiviral, antithrombotic, or anticoagulant. Fucoidans have been shown to inhibit the growth of a wide variety of tumor cells. See Itoh, H.; Noda, H.; Amano, H.; Ito, H. Anticancer Research 1995, 15, 1937-1947.

In another study fucans from Aschophyllum nodosum inhibited growth of smooth muscle cells, suggesting an anti-proliferative effect . See Logeart, D.; Prigeant-Richard, S.; Jozefonvicz, J.; Letourneur, D. European Journal of Cell Biology 1997, 74, 385-390.

Fucoidan extracted from Fucus vesiculosus was shown to exhibit immunomodulation properties See Kim, M. H.; Joo, H. G. Immunology Letters 2008, 115, 138-143.

The compositions and structural complexity of fucoidans from different brown seaweeds can vary considerably. The amount and the position of the sulfated groups along the backbone play a significant role in the functional properties of fucoidans. Therefore the properties of brown algae extracts and their corresponding fucoidans, perhaps responsible for much of the extract biological activity, cannot be predicted from one extract to another.

The present invention describes the unexpected discovery that extracts of Fucus vesiculosus brown algae exhibit exceptional stimulatory effects for endothelial cells migrations and pro-angiogenic properties providing high potential in wound healing properties.

### Extract

Extract means for purposes of this application an extract of Fucus vesiculosus. An extract of Fucus vesiculosus, may be obtained by extraction methods known to those skilled in the art. The extraction may be obtained by aqueous extraction or extraction with a C₁-C₄ alcohol or a water/alcohol mixture, wherein said alcohol may be a C₁-C₄ alcohol. Preferably, the extract is an aqueous extraction such as an alcohol/water blend. Furthermore the extraction procedure may include the extraction of Fucus vesiculosus with aqueous media containing both non-ionic surfactants such polyethyleneglycol (PEG) and the like and C₁-C₄ alcohols.

The amount of water used in the extraction medium will vary from about 75 wt. % to about 100 wt. %. Most typically water will make up about 85 to 99 wt. % of the extraction media.

C₁-C₄-alcohols are for example methanol, ethanol, propanol, isopropanol, butanol or mixtures thereof.

The C₁-C₄ alcohol when present in the extraction media will vary from about 1 to about 8 wt. %. Most typically the alcohol will vary from about 2 to about 6 wt. % and the wt. % is based on the total extraction media (excluding the amount of Fucus vesiculosus to be extracted.)

The wt. % of the Fucus vesiculosus subject to extraction in the media will vary from about 0.5 to about 15 wt. %, more typically about 1 to about 12 wt. % of the extract media and the weight % is based on the total extraction media.

The Fucus vesiculosus extract media is aqueous and water makes up about 75 wt. % to about 100 wt. %, preferably about 85 wt. % to about 99 wt.% of the extract media and the weight % is based on the total extract media.

The Fucus vesiculosus is typically ground to increase the surface area before extracting.

### Subject

Subject means for purposes of this application a mammal, for example a dog, horse, pig or human. Preferably, the subject is a human.

Effective Amount A effective amount administered to a subject in need means the concentration of the Fucus vesiculosus extract based on the total weight of the composition and will vary from about 0.01% to about 5% by weight of the total composition, preferably between about 0.02% to about 3.5% and most preferably between about 0.030% to about 3.5% based on the total weight of the composition.

### Wounds

The wound can be, e.g., a thermal, chronic, acute or surgical wound.

Preferably the wound is a chronic wound and exhibits a different healing profile from normal acute wounds in that they generally remain in an inflamed state for protracted periods of time. Non-healing wounds can most commonly be observed amongst people with diabetes, venous stasis disease, and in those patients who are immobilized.

### Wound Active Healing Agents

The composition comprising Fucus vesiculosus extract may further include at least one wound active healing agent. For example these wound active healing agents may be selected from the group consisting of preservatives, stabilizing agents, anti-oxidants, antimicrobials, antibiotics, trophic factors, growth factors, extracellular matrices (ECMs), cytokines, enzymes, enzyme inhibitors, anti-inflammatory agents, defensins, polypeptides, anti-infective agents (including antimicrobials, antivirals and antifungals), buffering agents, vitamins and minerals, analgesics, anticoagulants, coagulation factors, vasoconstrictors, vasodilators, diuretics, collagenases, a gel-forming or absorbent biocompatible polymer and mixtures thereof.

### Wound Care Products

Wound care products for purposes of this application means a topical dressing comprising a composition of an effective amount of Fucus vesiculosus and may be selected from the group of dressings consisting of a bandage, a membrane, synthetic or biological hydrogels, hydrocolloids, films, foams, a gauze, a dermal patch, an adhesive tape, a skin substitute, a spray, rope, ribbon or a sheet.

The effective amount of Fucus vesiculosus on or in a topical dressing will vary in such a way as the extract will be be released to the wound at about 0.01% to about 5% by weight of the total composition, preferably between about 0.02% to about 3.5% and most preferably between about 0.030 % to about 3.5% based on the total weight of the composition.

For example, the wound bed may be treated with a large variety of topical dressings, such as gels, pastes, powders, fibers or gauze, and synthetic or biological (acellular or cellular) hydrogels, hydrocolloids, films or foams. Depending on the wound type, compression therapy may be used concurrently with the appropriate topical treatment. Any of the dressings may further contain the extract of Fucus vsiculosus.

While lacking direct biological activity, synthetic dressings promote healing by fulfilling a multitude of functions. Hydrogel (Aquasorb®, Ashland; DuoDerm®, Convatec) and hydrocolloid (Tegasorb®, 3M; DuoDerm® CGF, Convatec; Hydrocoll®, Hartmann) dressings are suitable to maintain a moist wound environment necessary for healing. Polyurethane foams, pads or ribbons, salts of alginic acids, and other gellable polysaccharides, are used for their high absorptive capacity in the management of exudative wounds (Algisite®, Smith & Nephew; Lyofoam®, Molnlycke Heath Care; Curasorb®, Kendall; Spyrosorb®, BritCair; Multidex Maltodextrin®, DeRoyal; Aquacel®, Convatec). Other dressing combinations include multilayer systems that combine foams with a permeable, water-proof inner layer backing, and non-adherent composite dressings with a superabsorbant polymer layer (SAP) and a water-proof, permeable backing, that gives high capacity under compression (Enluxtra®, BASF; Xtrasorb®, Derma Sciences). The application of SAPs are used to sequester exudate into the dressing to keep the wound moist but without maceration.

Film dressings are generally used for clean, dry wounds with minimum exudate to provide exterior protection. They can be breathable or adhesive, and may even provide microbial protection (Tegaderm®, 3M; Dermafilm®, Dermarite Industries; Bioclusive®, Systagenix).

Biological dressings are used in direct contact with the wound, and are biocompatible and biodegradable. They are based on biomaterials, decellularized animal or human tissue, or cellular skin substitutes. Their purpose is to provide a matrix scaffold for cell proliferation and migration, combined with a potent biological effect. A natural, biologically active matrix can be very effective stimulant for healing.

Many biological dressings include reconstituted collagen films and sponges from bovine or other sources, alone (Puracol®, Medline Industries; Suprasorb®, Lohmann & Rauscher) or in combination with other biopolymers (collagen-ORC, Promogran®, Systagenix ; collagen-alginate, Fibracol®, Systagenix; collagen-chondroitin sulphate, Integra®, Integra Life Sciences; collagen-gelatin-CMC, Biostep®, Allegro Medical). The stimulating role of collagen, the major protein in skin, in the different phases of wound healing is well understood and documented. Other biopolymers employed for wound dressings include hyaluronic acid (Hyalomatrix®, Anika Therapeutics) and keratin (Keramatrix®, Keraplast Technologies). New products based on other extracellular matrix (ECM) proteins components, fibronectin (composite skin substitute with keratinocyte and fibroblast cells, HP804-247, Healthpoint) and vitronectin (VitroGro®, Tissue Therapies), are currently in clinical trials in the US. In some cases the dressings contain silver for additional antimicrobial protection (Aquacel® AG, Convatec; Promogran Prisma® and Fibrocol® Plus, Systagenix).

Decellularized tissue products include Oasis® (porcine intestinal submucosa, Healthpoint), Alloderm® (human cadaver skin, Lifecell), or Medeor® Matrix (porcine dermis, DSM). The advantage of decellularized products is that they contain all the active components of a normal ECM scaffold, providing the necessary cell signaling and direction for new ECM production by resident fibroblasts. However, these products carry a higher risk of viral contaminants and are potentially antigenic.

Advanced wound dressings are bioengineered skin substitutes where collagen scaffolds are cultured with human allogeneic keratinocyte and/or fibroblast cells. Only two products have been so far commercially approved for chronic wounds, Apligraf® (Organogenesis) and Dermagraft® (Organogenesis). The addition of new keratinocytes and fibroblasts to the wound increases the rate of healing by providing a natural source of growth factors and cytokines to augment wound healing. However, these treatments come at a very high cost.

Another approach is the topical application of growth factors, where the only commercially approved product is platelet-derived growth factor (PDGF), (Regranex® or Becaplermin®, Heathpoint). This product has been shown to increase healing rate by increasing fibroblast proliferation and ECM deposition. Other growth factors are in clinical trials (IGF and Epidemal Growth Factor (EGF), Tissue Therapies).

### EXAMPLES

The present invention is further demonstrated by the way of the following examples, which should not be considered limiting. Unless otherwise stated, the proportions given in any Examples herein are expressed as percentages by weight. The temperature is in degrees Celsius and the pressure is atmospheric pressure.

### Aqueous Extractions

Fucus vesiculosus biomass was obtained from commercial suppliers and various extractions of such biomass were made as shown below.

1.5 wt. % coarse ground Fucus vesiculosus biomass (wt. % of total extraction media) is added to 95 wt.% deionized water and 5 wt. %propanol, and mixed overnight at room temperature. The resulting mixture is coarse filtered through filter socks, then fine-filtered through stacked-disk filters with diatomaceous earth, followed by canister filters to a final pore size of 0.22 microns.

### Polyethylene Glycol (PEG) Extractions

10 wt.% (wt. % based on total extraction media) coarse ground Fucus vesiculosus biomass was added to 90% of a 15% PEG solution and 5 wt. % propanol, and mixed overnight at room temperature. The resulting mixture was coarse-filtered through filter socks, then fine-filtered through stacked-disk filters with diatomaceous earth, followed by canister filters to a final pore size of 0.22 microns.

An aqueous extract of Fucus vesiculosus (1%) was further diluted in PBS media to concentrations of 2.5, 5, 10, 20 and 30 mg/mL and used in migration and angiogenesis assays.

### 2D Migration Assay

HMVEC cells (human microvascular endothelial cells) are seeded on NUNC* 24-well plates at a density of 210⁵ cells/well, and cultured to confluence in 5% Bovine Calf Serum (BCS). 24 hours post-plating, the settled cell monolayers are wounded with a fire-polished Pasteur pipette, by making a straight, narrow, vertical scratch wound down the middle of the wells. The media is aspirated and the wells are washed twice with Phosphate buffered saline (PBS), and fed with fresh culture media containing the active to be tested (made up in 1% BCS media). Control wells contain only the basal serum free media. FGF (10 ng/mL) is added to media as a positive control. The migratory responses of the cells to the mechanical injury are monitored over 12 hours. The cells are placed onto the climate-controlled microscope stage (Axiovert®), and wound closure is quantified over time through time-lapse imaging using computer-assisted digital imaging under 10 magnification. The wound sizes are measured in pixels at the time of injury and at 1 hour, 6 hours, and 12 hours post-injury, using ImageJ software (available from NIH). Relative cell motility is calculated by comparing the change in area covered by cells in the same sized viewing field over the same period of time for different treatment conditions. The ratio of the wound area at different times to the initial wound area at time 0, gives the percent wound closure for that time period.

* NUNC=poly-N-isopropylacrylamide grafted to polystyrene by electron beam polymerization.

In vitro Testing of Extract

### 2D Migration Assay

HMVEC cells (human microvascular endothelial cells) are seeded on NUNC 24-well plates at a density of 210⁵ cells/well, and cultured to confluence in 5% BCS. 24 hours post-plating, the settled cell monolayers are wounded with a fire-polished Pasteur pipette, by making a straight, narrow, vertical scratch wound down the middle of the wells. The media is aspirated and the wells are washed twice with PBS, and fed with fresh culture media containing the active to be tested (made up in 1% BCS media). Control wells contain only the basal serum free media. FGF(fibroblast growth factor) (10 ng/mL) is added to media as a positive control. The migratory responses of the cells to the mechanical injury are monitored over 12 hours. The cells are placed onto the climate-controlled microscope stage (Axiovert), and wound closure is quantified over time through time-lapse imaging using computer-assisted digital imaging under 10 magnification. The wound sizes are measured in pixels at the time of injury and at 1 hour, 6 hours, and 12 hours post-injury, using ImageJ software (available from NIH). Relative cell motility is calculated by comparing the change in area covered by cells in the same sized viewing field over the same period of time for different treatment conditions. The ratio of the wound area at different times to the initial wound area at time 0, gives the percent wound closure for that time period.

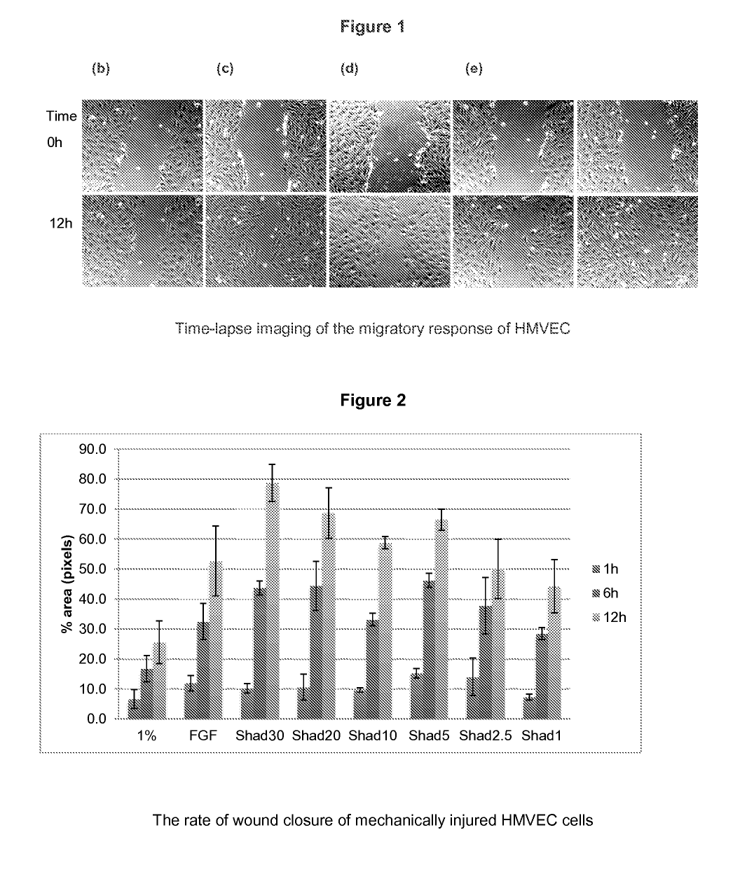

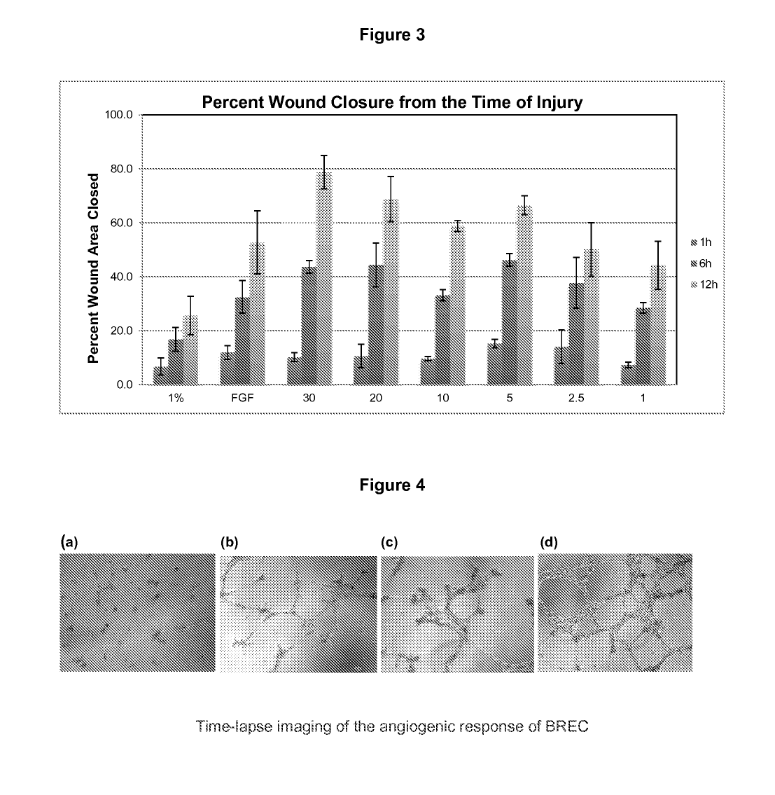

### See FIGS. 1-3.

### Angiogenesis Assay

Matrigel protein (GFR, growth factor reduced protein from mouse sarcoma cells) is polymerized for 1 hour at 37° C. in 8-well chamber slides (200 ml/well). BREC cells (bovine retinal endothelial cells) are pre-labeled with tetramethylrhodamine dextran sulfate dyes. Pre-labeled BREC cells (510⁴ cells/well) are plated within the layer-on-layer 3D Matrigel construct and incubated for 18 hrs in the presence of the active. Growth factors VEGF (Vascular Endothelial Growth Factor) and FGF are used as positive control (10 ng/ml). Tube formation is observed at 18 hrs post-plating. Images are taken using a Zeiss Axiovert 200 M microscope (5 and 10 magnification) equipped with a Hamamatsu (Orca ER) camera and a mercury fluorescence lamp (X-Cite), and analyzed using ImageJ. Sprouting is counted after staining in pixels/microns with either the image software analysis or and manually. Tube formation and their network are quantified by the number of tubes, number of nodes (>4 cells in width), and total tube length. See FIG. 4.

1. A method of promoting wound healing comprising administering to a subject in need thereof an effective amount of a composition comprising Fucus vesiculosus extract.

2. The method according to claim 1, wherein the extract is a water extract, a water/alcohol extract or a water/polyethylene glycol extract.

3. The method according to claim 1, wherein the composition comprising Fucus vesiculosus extract is administered directly to the wound site.

4. The method according to claim 1, wherein the subject is a mammal.

5. The method according to claim 1, wherein the effective amount of the Fucus vesiculosus extract in the composition ranges from about 0.01% to about 5% by weight of the total composition.

6. The method according to claim 2, wherein the Fucus vesiculosus extract is a water extract or an aqueous/C₁-C₄ alcohol extract.

7. The method according to claim 6, wherein the Fucus vesiculosus extract media is aqueous and the water makes up about 75 wt. % to about 100 wt. % and the weight % is based on the total extract media.

8. The method according to claim 7, wherein the wt. % of Fucus vesiculosus subject to extraction in the media will vary from about 0.5 to about 15 wt. % where the wt. % is based on the total weight of the extraction media.

9. The method according to claim 1, wherein the extract is administered in an amount effective to enhance the rate of endothelial cell migration, angiogenesis and the repair of blood vessels.

10. The method according to claim 1, wherein the wound is a thermal, chronic, acute or surgical wound.

11. The method according to claim 1, wherein the composition comprising Fucus vesiculosus extract is a powder, gel, spray, liquid, emulsion, aerosol, foam or paste, a rope, a ribbon or a sheet.

12. The method according to claim 1, wherein the composition comprising Fucus vesiculosus extract further includes at least one wound active healing agent selected from the group consisting of preservatives, stabilizing agents, anti-oxidants, antimicrobials, antibiotics, trophic factors, growth factors, extracellular matrices (ECMs), cytokines, enzymes, enzyme inhibitors, anti-inflammatory agents, defensins, polypeptides, anti-infective agents (including antimicrobials, antivirals and antifungals), buffering agents, vitamins and minerals, analgesics, anticoagulants, coagulation factors, vasoconstrictors, vasodilators, diuretics, collagenases, a gel-forming or absorbent biocompatible polymer and mixtures thereof.

13. A wound care product comprising an effective amount of a composition comprising Fucus vesiculosus extract, wherein the wound care product is a bandage, a membrane, a dressing, synthetic or biological hydrogels, hydrocolloids, films, foams, a gauze, a dermal patch, an adhesive tape, a skin substitute, a spray, rope, ribbon or a sheet.

14. A method of promoting wound angiogenesis comprising administering to a subject in need thereof an effective amount of a composition comprising Fucus vesiculosus extract.
